(19) United States
(12) Patent Application Publication
(10) Pub. No.: US 2013/0251748 A1
(43) Pub. Date: Sep. 26, 2013

(54) USE OF OUTER MEMBRANE PORIN K36 PROTEIN (OMPK36) IN TREATMENT/PREVENTION/DIAGNOSIS OF ENTEROBACTERIACEAE INFECTION

(71) Applicants: Leung-Kei Siu, Taipei City (TW); Feng-Yee Chang, Taipei City (TW); Yung-Chung Lin, Taipei City (TW); Chang-Phone Fung, Taipei City (TW); Yip-Mei Liu, Taipei City (TW); Jiun-Han Chen, Taipei City (TW); Yu-Kuo Tsai, Hsinchu City (TW); Pele Choi-Sing Chong, Miaoli County (TW); Chih-Hsiang Leng, Taipei City (TW); Shih-Jen Liu, Taichung City (TW); Hsin-Wei Chen, Taipei City (TW)

(72) Inventors: Leung-Kei Siu, Taipei City (TW); Feng-Yee Chang, Taipei City (TW); Yung-Chung Lin, Taipei City (TW); Chang-Phone Fung, Taipei City (TW); Yip-Mei Liu, Taipei City (TW); Jiun-Han Chen, Taipei City (TW); Yu-Kuo Tsai, Hsinchu City (TW); Pele Choi-Sing Chong, Miaoli County (TW); Chih-Hsiang Leng, Taipei City (TW); Shih-Jen Liu, Taichung City (TW); Hsin-Wei Chen, Taipei City (TW)

(73) Assignee: NATIONAL DEFENSE MEDICAL CENTER, TAIPEI CITY (TW)

(21) Appl. No.: 13/545,492

(22) Filed: Jul. 10, 2012

(30) Foreign Application Priority Data
Mar. 23, 2012 (TW) 101110192

(51) Int. Cl. A61K 39/108; A61P 31/04; G01N 21/76; A61K 39/112; G01N 33/569; G01N 21/64

(52) U.S. Cl. 424/257.1; 435/7.32; 435/7.35; 435/7.37; 424/259.1; 424/258.1

(57) ABSTRACT

The present invention relates a method for vaccinating a mammal to produce an antibody against Enterobacteriaceae infection caused by Klebsiella pneumoniae, Salmonella typhi, or E. coli in central nervous system and/or peripheral blood circulation, which comprises administering an effective amount of an OmpK36/homologues or its derivatives to the mammal.

### BACKGROUND OF THE INVENTION

1. Field of the Invention

The present invention relates to a method for treating and/or preventing or diagnosing infection of central nervous system and/or peripheral blood circulation in a mammal caused by Klebsiella pneumoniae, Salmonella typhi, E. coli, which comprises administration to the mammal an effective amount of an OmpK36/homologue(s) or its derivatives to the mammal.

2. The Prior Arts

Klebsiella pneumoniae is a rod-shaped, gram negative bacterium of the Enterobacteriaceae family. It is an important community and hospital-acquired bacterial pathogen with extended spectrum beta-lactamase (ESBL) that is commonly found responsible for drug resistant characteristics. Klebsiella pneumoniae frequently causes severe diseases, such as pneumoniae, urinary tract infections. In immunity compromised patients, Klebsiella infection complication may be associated to symptoms of peripheral blood circulation (bacteremia), central nervous system (meningitis), and endophthalmitis that can result in blindness. These bacterial infections can be purulent infection, such as meningitis, brain abscess, subdural abscess, and epidural abscess. In general, bacteria invasion is caused by local infection and spread systematically throughout to the body. Bacteria can be delivered via the blood system from the infection site to the central nervous system.

In recent years, Klebsiella pneumoniae has been reported as a common factor of cryptogenic liver abscess. Similar survey results have also been reported in North America and Europe. Currently it is observed that the virulent factor causing cryptogenic liver abscess have a common characteristic of unique capsular polysaccharides (CPS) (Advances in Therapy, 24 (3), p. 589-593, May 2007). Reports have also shown that capsular polysaccharides are major determinant of its pathogenicity (Infection and Immunity, 43(1), p. 440-441, January 1984), because the capsule can protect the bacteria from destroy of phagocyte and complements and the capsule is composed of complex polysaccharides. It is already known that there are 77 serotype of capsular antigen, among which K1 and K2 serotype capsular antigen have been found to be lethal to mouse model of abdominal inflammation. Thus, K1 and K2 are recognized as most lethal serotype. Besides K1 and K2, other serotype strains are almost non-lethal or non-pathogenic (Infection and immunity, 40 (1), p. 56-61, April 1983).

Klebsiella pneumonemiae mainly contains three types of outer membrane porins (Omps), including OmpK35 (Antimicrobial agents and chemotherapy, 42 (7), p. 1636-1640, July 1998), OmpK36 (Journal of bacteriology, 181 (9), p. 2726-2732, May 1999) and OmpK37 (The Journal of antimicrobial chemotherapy, 46 (2), p. 273-277, August 2000). The amino acid sequences of these outer membrane porins are homologous to the amino acid sequences of E. coli OmpF, OmpC and OmpN. The abovementioned outer membrane porins function as transporter of extracellular substrates across cell membrane, such as ferric ion, nutrients and antibiotics. Studies have shown that OmpK35 and OmpK36 play an important role in antibiotic resistance. Klebsiella pneumoniae has been found with increased resistance to many kinds of antibiotics (such as cephalosporins) and reduced susceptibility to antibiotics (such as cefoxitin, third generation of cephalosporin, monobactams and fluoroquinolones) when Omp porins are deleted in the cell. Moreover, Klebsiella pneumoniae isolated from the clinical specimens without Omp proins on the extracellular membrane (Antimicrobial agents and chemotherapy, 41 (3), p. 563-569, March 1997) have been associated with meningitis of central nervous system infection caused by Klebsiella pneumoniae (International journal of antimicrobial agents, 30 (5), p. 385-389, November 2007). OmpK36 is a highly conserved protein in the evolution of Enterobacteriaceae (Structure, 15; 7(4):425-34, April 1999); therefore, the present invention has the same function as OmpK36 of various Enterobacteriaceae that is commonly recognized by the community of this field. Alterti S et al. have found that degree of similarity of OmpK36 sequences of Klebsiella pneumoniae or Salmonella typhi is high after alignment comparison (Infection and immunity, 63 (3), p. 903-910, March 1995).

In the past several types of cellular constituents of Klebsiella pneumoniae (including capsular polysaccharides, lipopolysaccharides (LPS), cellular toxins, and whole lysates) or other antigens have been developed as vaccine against Klebsiella. High risks have been found to associate with these types of vaccines comprising constituents, because they may cause toxic response such as erythema or various kinds of pyogenic symptoms. In addition, there are 77 serotypes of capsular polysaccharides. It is difficult to use a single or several kinds of antigens to encompass all these types of serotypes. Although there are Klebsiella pneumoniae DNA vaccine studies in recent years, difficulty to control in vivo expression and toxicity of DNA vaccine are of great concerns. Therefore, there remain high risks if the DNA vaccine is to be applied in human clinically. Moreover, there is no report of application(s) regarding outer membrane porins or their derivative(s) in treatment and/or prevention and/or diagnosis of central nervous system and/or peripheral blood circulation infection.

### SUMMARY OF THE INVENTION

An objective of the present invention is to provide a novel, safe and effective method for vaccinating a mammal (including human) of high risk groups such as diabetic patients to produce antibodies against Klebsiella pneumoniae, Salmonella typhi, E. coli and other Gram-negative bacteria for control diseases of central nervous system and/or peripheral blood circulation system.

Another objective of the present invention is to provide a method for the treatment and/or prevention and/or diagnosis of bacterial infection caused by Klebsiella pneumoniae, E. coli or other Gram-negative bacteria in central nervous system and/or peripheral blood circulation in a mammal, which comprises administering an effective dose of an outer membrane porin K36 protein (OmpK 36)/homologue(s) or its derivatives to the mammal

A further objective of the present invention is to provide a method for detecting or diagnosing bacterial infection caused by Klebsiella pneumoniae, Salmonella typhi, or E. coli in central nervous system and/or peripheral blood circulation in a mammal, which comprises coating a first specific anti-OmpK36 antibody onto a matrix surface that can immunospecifically bind to OmpK36 molecular in blood or OmpK36 on the bacterial cellular membrane; adding a specimen from peripheral blood circulation and/or central nervous system to the matrix; adding a second anti-OmpK36 antibody with a label; and detecting the binding of the anti-OmpK36 antibodies to the OmpK36 molecule or OmpK36 on the bacterial cellular membrane, wherein the binding results demonstrates that the mammal may suffer from the bacterial infection in the central nervous system and/or peripheral blood circulation.

The present invention of outer membrane porin OmpK36 derived from Enterobacteriaceae family, such as Klebsiella pneumoniae, Salmonella typhi, or Escherichia coli, can be applied as in vitro diagnostics of bacteria and diseases to detect the presence of Klebsiella pneumoniae or other Enterobacteriaceae. The invention can also be applied in screening candidate compounds that interfere life cycle of Klebsiella pneumoniae or suppress infection of Klebsiella pneumoniae.

### BRIEF DESCRIPTION OF THE DRAWINGS

FIG. 1 exhibited the results of SDS-PAGE of OmpK36 protein of the present invention.

FIG. 2 showed the results of Western Blotting of OmpK36 protein of the present invention.

FIG. 3 demonstrated the percent of survival of mouse injected with recombinant OmpK36 then challenged with Klebsiella pneumoniae NVT-1001 (about 1×10³ cfu).

FIG. 4 demonstrated the percent of survival of mouse injected with recombinant OmpK36 then challenged with Klebsiella pneumoniae NVT-1002 (about 1×10⁵ cfu).

FIG. 5 exhibited the cytotoxicity of OmpK36 to the Hep-G2 cells.

### DETAILED DESCRIPTION OF THE PREFERRED EMBODIMENT

The examples of the present invention provide characteristics, purposes and advantages of the invention. The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

Unless further explained, the terms “a, an, the” are intended to mean singular or plural.

Unless further defined in the examples, all components, reaction conditions are approximate value and the variation can be in the range of ±5%. Therefore, unless a contradiction, all values can be regarded as an approach adjustment to obtain desired reaction results or properties.

The “central nervous system” in the present invention intends to that part of the nervous system that consists of the brain and spinal cord; the “peripheral blood circulation” intends to mean blood in the systemic circulation.

The “bacteria infection” in the present invention is intended to mean infections caused by Gram-negative bacteria, especially those infections caused by Enterobacteriaceae or other Gram-negative bacteria. Preferably, those bacterial infections caused by Escherichia coli, Klebsiella pneumoniae, or Salmonella typhi.

The outer membrane porin OmpK36 is a group of protein containing highly conserved sequence in the process of evolution (Structure, 15; 7(4):425-34, April 1999). Therefore, the present invention has the same function as OmpK36 of various Enterobacteriaceae that is commonly recognized by a skilled person in the art called as OmpK36 homologues in the present invention. Alberti S et al. have found that OmpK36 sequence of Escherichia coli, Klebsiella pneumoniae, and Salmonella typhi have high degree of similarity after alignment comparison (Infection and immunity, 63 (3), p. 903-910, March 1995). Preferably, the OmpK36 and its homologue(s) are obtained from Escherichia coli, Klebsiella pneumoniae, or Salmonella typhi. The outer membrane porin OmpK36 or its homologue(s) in the present invention is intended to mean any OmpK36 obtained from any Gram-negative bacteria or any recombinant OmpK36 protein. OmpK36 or its homologues is a structural protein rich in the outer membrane of Gram-negative bacteria. The “OmpK36 derivatives” are intended to mean any modification of OmpK36/homologues. For example, a recombinant OmpK36 with the same function(s) as OmpK36 is a derivative of OmpK36. Preferably, OmpK36, its homologue(s) or derivative(s) is obtained from Enterobacteriaceae or other Gram-negative bacteria.

According to the present invention, the invention relates to administrate an effective amount of OmpK36 protein/homologue(s) or its derivative(s) to a mammal (including human or non-human mammals) for treatment of central nervous system and/or peripheral blood circulation infection. The administration OmpK36/homologue(s) of the present invention or its derivative(s) can be conducted via any of common practice. Administration of the present invention OmpK36/homologue(s), or its derivatives can be in forms of prior art and fast and/or modified delivery format, such as tablet (non-coated or coated, such as anti-acid, slow released, non-soluble coating or coating for control release of the present invention), fast oral disintegrating tablets, thin films/lyophilisate tablet, capsule (such as hard or soft capsule), sugar coated tablet, granules, pills, powder, emulsion, suspension, aerosol or solution.

Parenteral administration includes intravenous drip or infusion, subcutaneous, intraperitoneal or intramuscular injection, pulmonary administration, e.g. by inhalation or insufflation, or intrathecal or intraventricular administration. Administration forms suitable for parenteral administration include injection and infusion preparations in the form of solutions, suspensions, emulsions, lyophilisates or sterile bacterial powder.

It is preferred that oral or non-intestinal administration of the OmpK36 protein, especially oral and intra-venous injection is preferred, and intra-venous dose form is most preferred, for example for acute central nervous system infection.

OmpK36/homologue(s) and its derivative(s) of the present invention can be applied or converted into a suitable pharmaceutical composition, wherein inert, non-toxic excipients can be mixed with the effective amount of the present invention to meet treatment requirements. The excipients can include carriers (for example micro-crystal cellulose, lactose, mannose), solvent (for example, liquid polyethylene glycol), emulsion and dispersion or wetting agent (for example sodium dodecyl sulfate, polyoxysorbitan oleate), binder (for example polyvinylpyrrolidone), synthetic and natural polymers (for example albumin), stabilizer (for example antioxidant such as, for example, ascorbic acid), colorant (for example inorganic colorant such as, for example, iron oxides) and masking tastes and/or odors.

The administration form of the present invention comprises of 0.0001% to 10% of OmpK36 protein/homologue(s) or its derivatives, on a weight basis, preferably in the range of 0.5 to 5% of OmpK36, on a weight basis. Time for administration of the pharmaceutical composition of the present invention depends on seriousness of the disease and condition of individual patient. Doctors should determine enough time for treatment when administration the pharmaceutical composition of the present invention.

The present invention further provides a novel vaccine against bacterial infection in the central nervous system and/or peripheral blood circulation, and the vaccine comprises the outer membrane porin (OmpK36 protein)/homologue(s) or its derivative(s) to immunize a mammal to produce antibody against bacterial infection. In other words, the present invention provides a method for vaccinating a mammal to produce an antibody against bacterial infection in central nervous system and/or peripheral blood circulation, which comprises administering to said mammal an effective amount of an OmpK36 protein/homologue(s) or its derivative(s). On the basis of foundation of OmpK36 can treat and/or prevent and/or diagnose bacterial infection in central nervous system and/or peripheral blood circulation, the OmpK36 was introduced to an animal for vaccination. The present invention had discovered that OmpK36 can induce specific antibodies production.

The antibody of the present invention can be produced by known prior art or technology of similar field. Anti-OmpK36 polyclonal antibody and monoclonal antibody can be produced by any technology and method known in the art, for example, administration of OmpK36 to a host animal (including, but not limited to, rabbit, mouse, mice) to induce synthesis of polyclonal antibodies. Depending on the host, various adjuvants can be used to enhance immunity response. The adjuvants include, but not limited to, Feund's adjuvant, mineral gel (for example, aluminum hydroxide), surfactant (for example, lysolecithin), complex polyol (pluronic polyols, poly anions), peptide, hydrophobic emulsion, keyhole limpet hemocyanin, dinitrophenol, and potential human adjuvant (for example, bacilli Calmette-Guerin (BCG) and Corynebacterium parvum). These adjuvants are well-known in this technological field of the present invention.

The antibody of the present invention can be applied in quantitative analysis of OmpK36 in the specimen, using any serology and immunohistochemistry method known to those of skill in the art or described herein. Other antibody based methods useful for detecting protein gene expression include immunoanalysis (for example, enzyme-linked immunosorbent assay (ELISA) and radioimmunoassay (RIA)). Suitable antibody assay labels are known in the art and include enzyme labels, such as enzyme labeling (for example, glucose oxidase), radioactive labeling (for example, iodine (125I, 131I), carbon 14 (14C), sulfur (35S), indium (121In), Tritium (3H) and Technetium (99Tc)), chemiluminescence labeling (for example, luminol), fluorescence labeling (for example, fluorescein and ehodamine), and biotin.

Moreover, the present invention provides a method of detecting or diagnosing bacterial infection in central nervous system and/or peripheral blood circulation in a mammal. In one embodiment, the detection or diagnosis comprises: coating a first specific anti-OmpK36 antibody onto a matrix surface (for example, ELISA plates or magnetic nano-particles) that can immunospecifically bind to the OmpK36 molecular in blood or OmpK36 on the bacterial cellular membrane; adding a specimen from peripheral blood circulation and/or central nervous system to the matrix; adding a second anti-OmpK36 antibody with a label; and detecting the binding of anti-OmpK36 antibodies to the OmpK36 molecule or OmpK36 on the bacterial cellular membrane, wherein the binding results demonstrates that the mammal may suffer from the bacterial infections in the peripheral blood circulation and/or the central nervous system.

Based on the present invention, the first specific anti-OmpK36 antibody is coated onto the matrix surface using a method and commercial coating buffer known in the art and any appropriate matrix can be used in the method. The matrix is preferably ELISA plates or magnetic nano-particles. The anti-OmpK36 antibody can specifically bind to OmpK36 molecule in the blood or OmpK36 on the bacterial cell membranes. To allow the binding detectable, the anti-OmpK36 antibody specifically binding to OmpK36 molecule can be detected by using a second anti-OmpK36 antibody with a label. According to the present invention, the term “label” intends to a molecule or moiety having a property or characteristic which is capable of detection. A label may be directly detectable, as with radioisotopes, fluorophores or chemilumiphores; or a label may be indirectly detectable, as with haptens or polynucleotide tails. When indirect labels are used for detection or signaling purposes, they are used in conjunction with a signaling entity complex. A “signaling entity” is a molecule or moiety which provides the detectable property or characteristic. The signaling entity may be direct, as with a colloidal particle (e.g. colloidal gold or selenium); or it may be indirect, as with an enzyme (e.g. alkaline phosphatase, β-galactosidase or horseradish peroxidase). Indirect signaling entities may require additional components, e.g. substrate, as is well known in the arm. The “signaling entity complex” includes a signaling entity conjugated to specific binding partner, such as an antibody or polynucleotide. Such conjugates may be prepared according to any known method of conjugation.

According to the invention, the binding of the anti-OmpK36 antibody to the OmpK36 molecule in peripheral blood or central nervous system can be used to detect the existence of the OmpK36 molecule. If the OmpK36 molecule exists in the peripheral blood or central nervous system of a subject, it represents that the subject may be infected by a bacteria with OmpK36 molecule.

### EXAMPLE 1

### Preparation of Recombinant OmpK36 Protein

Primer pairs of OmpK36F (gggaattccatatgcaccatcatcatcatcacatga aagttaaagtactg (SEQ ID NO:1)) and OmpK36R (ccgctcgaggaactggt aaaccaggcc (SEQ ID NO:2)) were used as primer pairs for amplification of OmpK36 DNA fragment. Amplified fragments were cut with restriction enzyme Nde I and xho I and inserted into the expression region of protein expression plasmid pET30a (purchased from Novagen) to construct pET30a-OmpK36 plasmid. The protein product is His-OmpK36 protein.

pET30a-OmpK36 plasmid was transformed into competent cell BL-21 (DE3) and the transformed cells were incubated in culture medium containing 50 μg/L kanamycin at 37° C. overnight. The transformed cells were grown in Luria-Bertani culture medium until OD₆₀₀ reached 0.7 to 0.9. Induction of protein expression was done by addition of IPTG (1 mM) at 37° C. for 4 hours. Cells were then collected by centrifugation at 9,000 g for 30 minutes. Bacterial cells were broken by sonication (Sonics Vibracell sonicator (sonicated at 25% power at 4° C. for 5 minutes) and dissolved in PBS containing 6M urea to obtain whole cell lysate.

Soluble proteins were mixed with Ni2+ gel (purchased from Amersham). His 6 on the protein was allowed to bind to the Ni2+ gel and then eluted with buffer (10 mM imidazole, 6M urea, PBS containing 10% glycerol). The large amount of imidazole in the protein solution was removed by dialysis for 4 hours using PBS containing 3M urea and 10% glycerol. Finally, the protein was dissolved in PBS containing 10% glycerol and endotoxin was removed using Pierce Detoxi-Gel (purchased from Thermo Scientific, Rockford, Ill.).

### Western Blotting

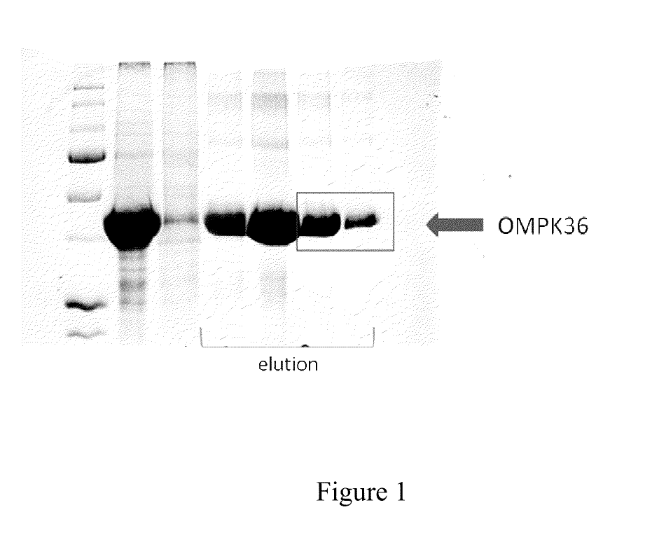

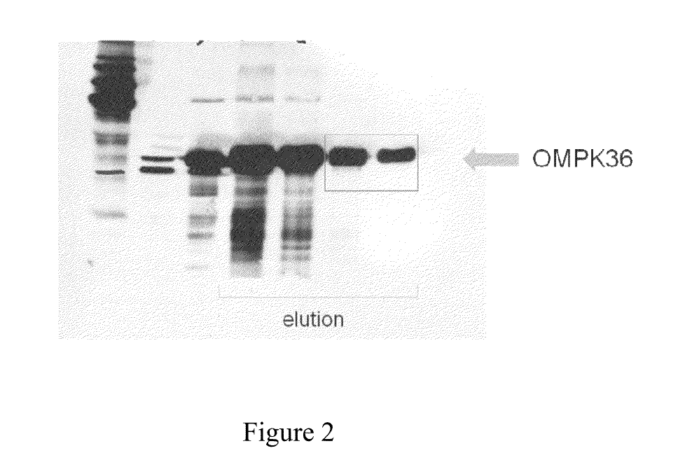

The whole cell lysate preparation as described above was subjected to 12% SDS-PAGE electrophoresis (2 μl/channel). The results were shown in FIG. 1. After electrophoresis, proteins in the gel were transferred to PVDF membrane (purchased from Milipore) and the PVDF membrane was immersed in 5% non-fat milk PBST buffer (PBS buffer containing 0.05% Tween 20) for 1 hour. The PVDF membrane was placed in 5% non-fat milk solution containing anti-His antibody (AbD Serotec, UK; 1:2500) at room temperature for 1 hour and washed with PBST buffer three times. The membrane was allowed to react with the horseradish peroxidase-conjugated secondary antibody (1:2500) at room temperature and then washed with TTBS buffer three times. Because the secondary antibody containing the horseradish peroxidase, ECL system (enhanced chemiluminescence diction kit, PerkinElmer Inc., MA, USA) was used to react with the membrane, chemiluminescence X-ray film. Referring to FIG. 2, purity of His-OmpK36 protein was confirmed with western blot (anti-His). Total protein concentration was quantified using Bradford analysis method (Bio-Rad).

### Quantification of Protein

His-OmpK36 concentration was determined by Bradford protein assay. His-OmpK36 protein solutions at various concentrations were mixed with Bio-Rad protein assay dye and allowed to react for 5 minutes. Protein concentration was measured using spectrophotometer at wavelength of 595 nm The responses of the standards were used to plot or calculate a standard curve. Absorbance values of unknown samples were then interpolated onto the plot for the standard curve to determine their concentrations.

### EXAMPLE 2

### Animal Experiments

Inbred male BALB/c mice (6 to 8 weeks old) were purchased from the National Laboratory Animal Center (Taipei, Taiwan) and were allowed one week to acclimatize before the experiment. Animal treatment and tests followed the guide for the Care and Use of Laboratory Animals published by Institutional Animal Care and Use Committees. These 6 to 8 week-old mice were divided into groups randomly and subjected to administration of purified recombinant OmpK36 (60 μg) subcutaneously. The injection formulation contained PBS supplemented with 10% glycerol (100 μl) and Freund's adjuvant (100 μl; Sigma). After two weeks, mice were immunized again with the same administration.

Mice immunized with recombinant OmpK36 were challenged with bacterial infection (about 1×10³ cfu or 1×10⁵ cfu), for example, virulent Klebsiella pneumoniae NVT-1001 (5.8×10², serotype K1) or NVT-1002 (2.4×10⁵ cfu, serotype K1). In general, infected mice will develop bacteremia and spread throughout whole body. The central nervous system of mice would be invaded at the end stage. 100 μl PBS was used as control group. Survival in mice and symptoms of mice were assessed two weeks post-administration.

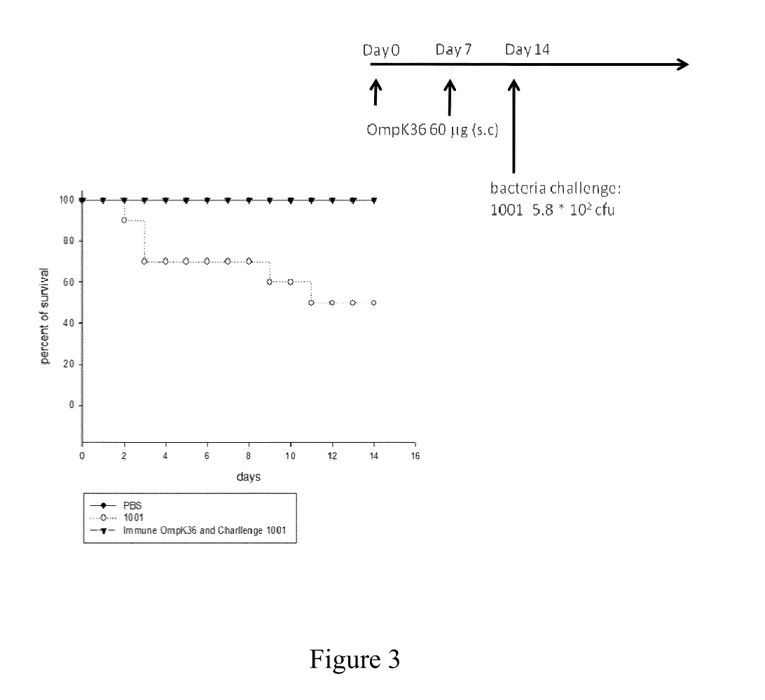

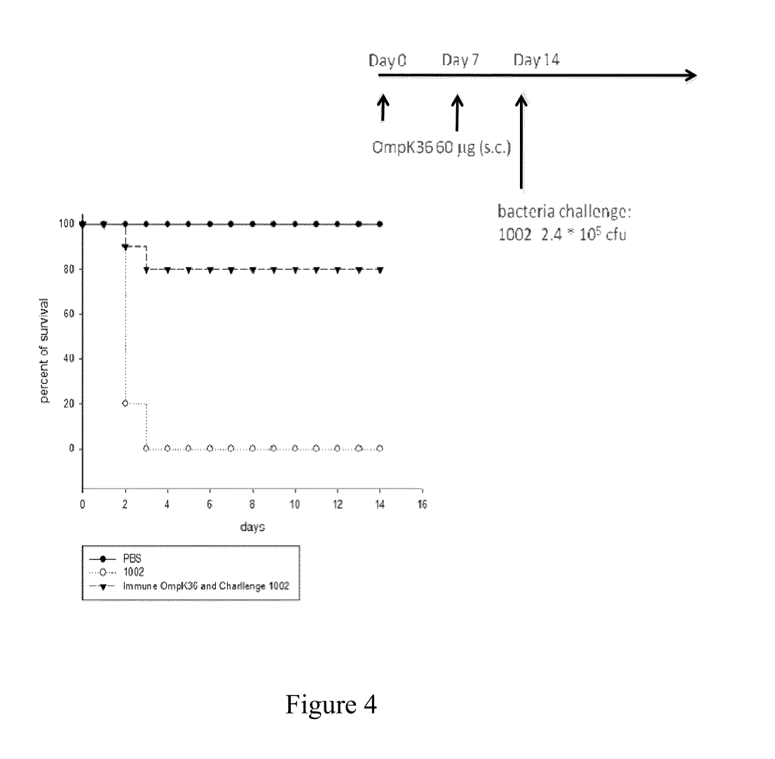

Referring to FIG. 3, OmpK36 prolongs the survival of the mice subcutaneously injected with OmpK36 then challenged with infection of Klebsiella pneumoniae NVT-1001. Death ratio of mice without OmpK36 administration was about 50% two weeks post-infection challenge of Klebsiella pneumoniae NVT-1001. On the contrary, mice received 60 μg OmpK36 injection all survived when underwent the same Klebsiella pneumoniae NTV-1001 infection challenge two weeks post-infection challenge. FIG. 4 showed higher survival percentage of mice received OmpK36 injection when challenged with Klebsiella pneumoniae NVT-1002. The control group (without OmpK36 treatment) mice all died 3 days after challenged with Klebsiella infection, whereas survival ratio was 80% in the mice received 60 μg OmpK36 injection when treated with the same infection challenge process. The results demonstrated the OmpK36 of the present invention could significantly enhance survival ratio of mice.

### EXAMPLE 3

### Cytotoxicity

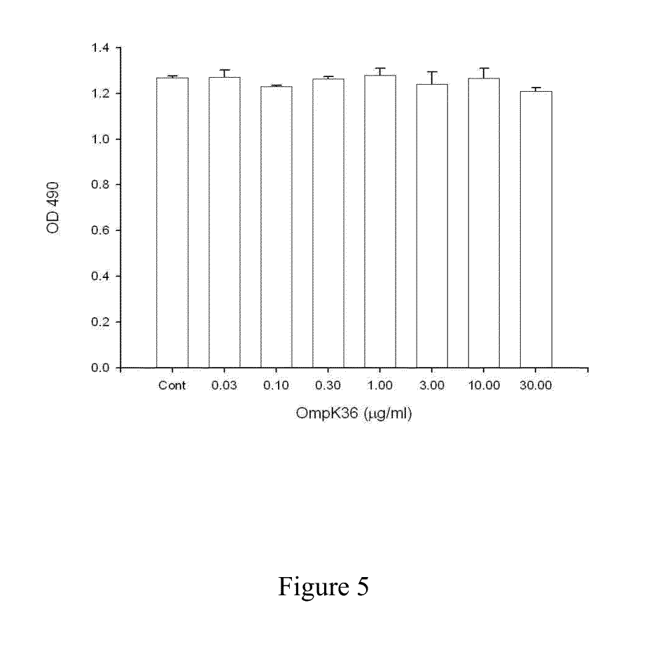

Recombinant OmpK36 (0.03 μg/ml-30 μg/ml) was incubated with the Hep-G2 hepatoma cells (purchased from American Type Culture Collection, ATCC) to evaluate its cytotoxicity. PBS containing 10% glycerol was used as control group. Cell survival was determined using Wallac Victor® multilabel counter (model 1420, Turku, Finland) and measured fluorimetrically at wavelength of 490 nm Referring to FIG. 5, recombinant OmpK36 at concentration up to 30 μg/ml showed no cytotoxicity to Hep-G2 cells. Although OmpK36 gene is an important virulence factor of Klebsiella pneumoniae, OmpK36 protein itself is not toxic to cells.

What is claimed is:

1. A method for vaccinating a mammal to produce an antibody against Enterobacteriaceae infection caused by Klebsiella pneumoniae, Salmonella typhi, or Escherichia coli in central nervous system and/or peripherial blood circulation, which comprises administering to the mammal an effective amount of an outer membrane porin K36 (OmpK36).

2. The method as claimed in claim 1, wherein the OmpK36 is obtained from Escherichia, Klebsiella pneumoniae or Salmonella typhi.

3. The method as claimed in claim 1, wherein the antibody is polyclonal antibodies.

4. The method as claimed in claim 1, wherein the mammal is human.

5. The method as claimed in claim 1, wherein the OmpK36 is a recombinant OmpK36 or its homologues.

6. The method as claimed in claim 1, wherein the OmpK36 is administrated at an amount of 0.0001% to 10% by weight of OmpK36.

7. The method as claimed in claim 1, wherein the OmpK36 is administrated at an amount of 0.5% to 5% by weight of OmpK36.

8. A method for the treatment or prevention of Enterobacteriaceae infection caused by Klebsiella pneumoniae, Salmonella typhi, or Escherichia coli in central nervous system and/or peripherial blood circulation in a mammal, which comprises administering to the mammal an effective amount of an outer membrane porin K36 (OmpK36).

9. The method as claimed in claim 8, wherein the OmpK36 is obtained form Escherichia coli, Klebsiella pneumoniae, or Salmonella typhi.

10. The method as claimed in claim 8, wherein the mammal is human.

11. The method as claimed in claim 8, wherein the OmpK36 is a recombinant OmpK36.

12. The method as claimed in claim 8, wherein the OmpK36 is administrated orally or via intravenous injection.

13. The method as claimed in claim 12, wherein the OmpK36 is administrated at an amount of 0.0001% to 10% by weight of OmpK36.

14. The method as claimed in claim 12, wherein the OmpK36 is administrated at an amount of 0.5% to 5% by weight of OmpK36.

15. A method for detecting or diagnosing bacterial infection caused by Klebsiella pneumoniae or Salmonella typhi in central nervous system and/or peripheral blood circulation in a mammal, which comprises coating a first specific anti-OmpK36 antibody onto a matrix surface that can immunospecifically bind to OmpK36 molecular in blood or OmpK36 on the bacterial cellular membrane; adding a specimen from peripheral blood circulation and/or central nervous system to the matrix, adding a second anti-OmpK36 antibody with a label; and detecting the binding of the anti-OmpK36 antibodies to the OmpK36 molecule or OmpK36 on the bacterial cellular membrane, wherein the binding results demonstrates that the mammal may suffer from the bacterial infection in the central nervous system and/or peripheral blood circulation; and wherein the OmpK36 is obtained from Klebsiella pneumoniae, Salmonella typhi, or Escherichia coli.

16. The method as claimed in claim 15, wherein the mammal is human.

17. The method as claimed in claim 15, wherein the matrix is ELISA plates or magnetic nano-particles.

18. The method as claimed in claim 15, wherein the label is radioisotopes, fluorophores or chemilumiphores.


SEQUENCE LISTING (2 sequences)

SEQ ID NO: 1 - DNA, 50 bp, Artificial Sequence
  5′-Primer for amplificating OmpK36
gggaattccatatgcaccatcatcatcatcacatgaaagttaaagtactg

SEQ ID NO: 2 - DNA, 27 bp, Artificial Sequence
  3′-Primer for amplificating OmpK36
ccgctcgaggaactggtaaaccaggcc